(19) United States
(12) Patent Application Publication
(10) Pub. No.: US 2019/0038784 A1
(43) Pub. Date: Feb. 7, 2019

(54) TAU PET IMAGING LIGANDS

(71) Applicant: Janssen Pharmaceutica NV, Beerse (BE)

(72) Inventors: Diederik Willem Elisabeth MOECHARS, ROTSELAAR (BE); Frederik Jan Rita ROMBOUTS, WILRIJK (BE); Joseph Elisabeth LEENAERTS, RIJKEVORSEL (BE); José Ignacio ANDRéS-GIL, MADRID (ES); Katleen FIERENS, ANTWERPEN (BE); Vladimir CHUPAKHIN, ANTWERPEN (BE); Guy Maurits R. BORMANS, ROTSELAAR (BE); Lieven Denis Herwig DECLERCQ, BIERBEEK (BE); Hartmuth KOLB, SAN DIEGO, CA (US); Wei ZHANG, SAN DIEGO, CA (US)

(21) Appl. No.: 16/073,992

(22) PCT Filed: Feb. 1, 2017
(86) PCT No.: PCT/EP2017/052143

§ 371 Date: Jul. 30, 2018

(30) Foreign Application Priority Data
Feb. 3, 2016 (EP) 16154123.0
Mar. 21, 2016 (EP) 16161466.4
Aug. 5, 2016 (EP) 16183009.6
Aug. 31, 2016 (EP) 16186603.3

(51) Int. Cl. A61K 51/04; C07B 59/00; A61K 9/00; C07D 471/04

(52) U.S. Cl. CPC A61K 51/0455; C07B 59/002; C07B 2200/05; C07D 471/04; A61K 9/00

(57) ABSTRACT

The present invention relates to novel, selective radiolabelled tau ligands which are useful for imaging and quantifying tau aggregates, using positron-emission tomography (PET). The invention is also directed to compositions comprising such compounds, to processes for preparing such compounds and compositions, to the use of such compounds and compositions for imaging a tissue or a subject, in vitro or in vivo, and to precursors of said compounds.

### FIELD OF THE INVENTION

The present invention relates to novel, selective radiolabelled tau ligands which are useful for imaging and quantifying tau aggregates, using positron-emission tomography (PET). The invention is also directed to compositions comprising such compounds, to processes for preparing such compounds and compositions, to the use of such compounds and compositions for imaging a tissue or a subject, in vitro or in vivo, and to precursors of said compounds.

### BACKGROUND OF THE INVENTION

Alzheimer's Disease (AD) is a neurodegenerative disease associated with aging. AD patients suffer from cognition deficits and memory loss as well as behavioural problems such as anxiety. Over 90% of those afflicted with AD have a sporadic form of the disorder while less than 10% of the cases are familial or hereditary. In the United States, about one in ten people at age 65 have AD while at age 85, one out of every two individuals are afflicted by AD. The average life expectancy from the initial diagnosis is 7-10 years, and AD patients require extensive care either in an assisted living facility or by family members. With the increasing number of elderly in the population, AD is a growing medical concern. Currently available therapies for AD merely treat the symptoms of the disease but not the underlying pathology causing the disease.

The hallmark pathological features in the brain of AD patients are neurofibrillary tangles which are generated by aggregates of hyperphosphorylated tau protein and amyloid plaques which form by aggregation of beta-amyloid peptide.

Though the most prevalent neurodegenerative disorder is AD, aggregated tau protein is also a characteristic of other neurodegenerative diseases known as “tauopathies”, which additionally but not exclusively include tangle-only dementia (TD), argyrophilic grain disease (AGD), progressive supranuclear palsy (PSP), corticobasal degeneration (CBD), Pick disease (PiD), and frontotemporal dementia and parkinsonism linked to chromosome 17 (FTDP-17). The heterogeneity of these disorders is closely related to the wide range of human tau isoforms and post-translational modifications. Tau aggregates may appear ultrastructurally as paired helical filaments (PHF), straight filaments (SF), randomly coiled filaments (RCF), or twisted filaments (TF); this variability translates into polymorphism. A correlation ofneurofibrillary tangles has been made with the level of cognitive impairment in AD and/or the chance of developing AD. However, diagnosis can still only be performed post-mortem by means of biopsy/autopsy. Examination based on history and statistical memory testing require clear evidence of impairment or dementia, and are often inaccurate or insensitive, and measurement of AB peptides and total tau proteins in cerebrospinal fluid by lumbar puncture is invasive and amenable to adverse effects. Apart from the intrinsic complexity of AD, the development of a cure has been hampered by the lack of reliable tools for early diagnosis, staging, and accurately monitoring disease progression. There is therefore still a need to identify a means to perform diagnosis and/or monitor disease progression. Imaging of tau aggregates may provide such means, particularly when anti-tau treatments emerge.

Positron Emission Tomography (PET) is a non-invasive imaging technique that offers the highest spatial and temporal resolution of all nuclear imaging techniques and has the added advantage that it can allow for true quantification of tracer concentrations in tissues. It uses positron emitting radionuclides for detection. Several positron emission tomography radiotracers have been reported so far for imaging of tau aggregates (for a review, see for instance Ariza et al. J. Med. Chem. 2015, 58, 4365-4382). There is still a need to provide selective, improved positron emission tomography radiotracers for imaging tau aggregates with a good balance of properties including, but not limited to, high affinity and selectivity towards tau aggregates, reversible binding, permeability, suitable brain pharmacokinetic profile, i.e. rapid distribution throughout the brain, rapid clearance, minimal non-specific binding, and synthetic accessibility.

### SUMMARY OF THE INVENTION

It is therefore an object of the present invention to provide compounds useful as tau PET radiotracers. Therefore, in one aspect, the present invention relates to a compound having the Formula (I)

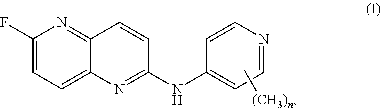

wherein
at least one atom is radioactive, and wherein the methyl substituent when present is bound to any available carbon atom in the pyridyl ring and n is 0 or 1, or a pharmaceutically acceptable salt or a solvate thereof.

In particular, the present invention relates to a compound of Formula (I′)

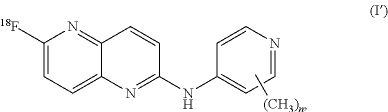

wherein the methyl substituent when present is bound to any available carbon atom in the pyridyl ring and n is 0 or 1, or a pharmaceutically acceptable salt or a solvate thereof.

In another aspect, the invention relates to precursor compounds for the synthesis of the compounds of Formula (I) or (I′), as previously defined. Thus, the present invention also relates to a compound of Formula (I-3), (I-A) and (P-1)

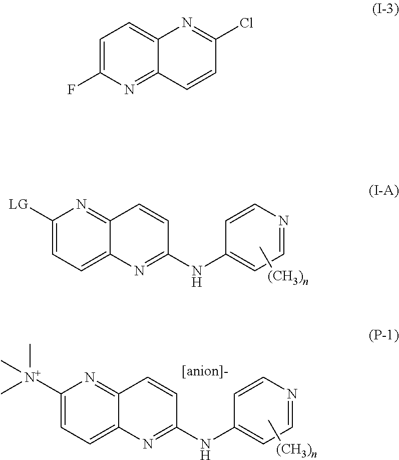

wherein when present, the methyl substituent is bound to any available carbon atom in the pyridyl ring, LG is a suitable leaving group, and [anion]⁻ is a suitable anionic counterion, or a pharmaceutically acceptable salt or a solvate thereof.

Suitable leaving groups are those that can be replaced by ¹⁸F and can be selected from the group consisting of trimethylammonium, chloro, bromo, nitro and 4-methylbenzenesulfonate (tosylate). Suitable anionic counterions include trifluoroacetate (—[OC(O)CF₃]⁻), an organic sulfonate (e.g. C₁₋₄alkyl sulfonate, or phenylsulfonate wherein the phenyl may be optionally substituted with a C₄₋₄alkyl, halo, or a nitro group) and tartrate. Particular examples of C₁₋₄alkylsulfonate include methanesulfonate (mesylate) and ethanesulfonate, and particular examples of phenylsulfonate include benzenesulfonate, 4-methylbenzenesulfonate (tosylate), 4-bromobenzenesulfonate and 4-nitrobenzenesulfonate. In particular, the anionic counterion is selected from trifluoroacetate (—[OC(O)CF₃]⁻), tosylate, and mesylate.

The invention also relates to the reference materials of compounds of Formula (I), corresponding to the corresponding non-radiolabelled compounds, herein referred to as compounds of Formula [¹⁹F]—(I)

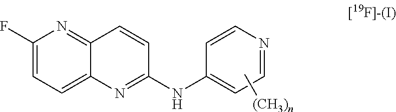

wherein the methyl substituent when present is bound to any available carbon atom in the pyridyl ring and n is 0 or 1, and the pharmaceutically acceptable salts and the solvates thereof.

The invention also relates to a pharmaceutical composition comprising a compound of Formula (I) or a pharmaceutically acceptable salt thereof and a pharmaceutically acceptable carrier or diluent. In particular, said pharmaceutical composition is a diagnostic pharmaceutical composition. Said pharmaceutical composition is in particular, a sterile solution. Thus, illustrative of the invention is a sterile solution comprising a compound of Formula (I) as described herein.

The invention further relates to the use of a compound of Formula (I) as an imaging agent. Exemplifying the invention is a use of a compound of Formula (I) as described herein, for, or a method of, imaging a tissue or a subject, in vitro or in vivo.

In particular, the invention relates to a compound of Formula (I) for use in binding and imaging tau aggregates in patients suffering from, or suspected to be suffering from, a tauopathy. Particular tauopathies are, for example, Alzheimer's disease, tangle-only dementia (TD), argyrophilic grain disease (AGD), progressive supranuclear palsy (PSP), corticobasal degeneration (CBD), Pick disease (PiD), and frontotemporal dementia and parkinsonism linked to chromosome 17 (FTDP-17). In particular, the tauopathy is Alzheimer's disease.

The invention further relates to a compound of Formula (I) for diagnostic imaging of tau aggregates in the brain of a subject, and to the use of the compound of Formula (I) in binding and imaging tau aggregates in patients suffering from, or suspected to be suffering from, a tauopathy. Particular tauopathies are, for example, Alzheimer's disease, tangle-only dementia (TD), argyrophilic grain disease (AGD), progressive supranuclear palsy (PSP), corticobasal degeneration (CBD), Pick disease (PiD), and frontotemporal dementia and parkinsonism linked to chromosome 17 (FTDP-17). In particular, the tauopathy is Alzheimer's disease.

The invention also relates to a method for imaging a tissue or a subject, comprising contacting with or providing or administering a detectable amount of a labelled compound of Formula (I) as described herein to a tissue, or a subject, and detecting the compound of Formula (I).

Further exemplifying the invention is a method of imaging a tissue, or a subject, comprising contacting with or providing to a tissue, or a subject, a compound of Formula (I) as described herein, and imaging the tissue, or subject with a positron-emission tomography imaging system.

Additionally, the invention refers to a process for the preparation of a compound of Formula (I′), or a pharmaceutically acceptable salt or a solvate thereof as described herein, comprising (a) the step of reacting a compound of Formula (P-1) or a pharmaceutically acceptable salt or a solvate thereof, as defined herein, wherein in particular, the anionic counterion is trifluoroacetate, with a source of fluoride ¹⁸F⁻ under suitable conditions or (b) the step of reacting a compound of Formula (I-A) or a pharmaceutically acceptable salt or a solvate thereof, as defined herein, with a source of fluoride ¹⁸F⁻ under suitable conditions. A suitable source of ¹⁸F⁻ is, for example 4,7,13,16,21,24-hexaoxa-1,10-diazabicyclo[8.8.8]hexacosane potassium fluoride-[¹⁸F](1:1) (also referred to as [¹⁸F]KF.K222). Suitable conditions include, those appropriate for nucleophilic substitution known in the art, for example, using DMF as solvent under conventional heating, for example at about 120° C., for a sufficient period of time to enable the reaction to proceed to completion.

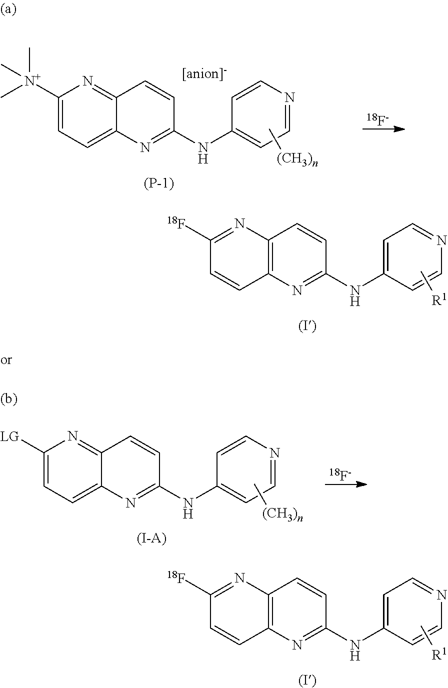

### BRIEF DESCRIPTION OF THE FIGURES

FIG. 1a shows immunohistochemistry images after incubation with AT8 antibody on a cryosection of human brain (AD) adjacent to the section shown in FIG. 1b.

FIG. 1b shows immunohistochemistry images after incubation with 4G8 antibody on a cryosection of human brain (AD).

FIG. 2 shows autoradiography images of [¹⁸F]Co. No. 1 on a cryosection of human brain (AD) adjacent to the section shown in FIG. 1b (left), displacement of the bound [¹⁸F]Co. No. 1 with 1 μM [¹⁹F]Co. No. 1 (middle), and displacement of the bound [¹⁸F]Co. No. 1 with 1 μM [¹⁹F]T808 (right).

FIG. 3 shows μPET time-activity curves for [¹⁸F]Co. No. 1 (FIG. 3a) and [¹⁸F]T807 (FIG. 3b) in the whole brain of three female Wistar rats. Baseline scan; pre-treatment experiment: cold Co. No. 1 or T807, 10 mg/kg injected subcutaneously 60 min prior to radiotracer injection and chase study: cold Co. No. 1 or T807, 1 mg/kg injected intravenously 30 min after radiotracer injection.

FIG. 4 shows baseline comparison of small animal PET time-activity curves of [¹⁸F]Co. No. 1 and [¹⁸F]T807 in the total brain of a Wistar rat.

FIG. 5 shows PET time-activity curves for [¹⁸F]Co. No. 1 (FIG. 5a) and [¹⁸F]T807 (FIG. 5b) in the whole brain, corpus callosum, cerebellum and entorhinal cortex and skull of a rhesus monkey.

FIG. 6 shows average whole brain % SUVmax curves of [¹⁸F]Co. No. 1 and [¹⁸F]T807 in, respectively, a female and a male rhesus monkey.

FIG. 7 shows μPET time-activity curves for [¹⁸F]Co. No. 1 (FIG. 7a) and [¹⁸F]T807 (FIG. 7b) in the whole brain, corpus callosum, cerebellum, enthorinal cortex and skull of a male rhesus monkey.

FIG. 8 shows average whole brain % SUVmax curves of [¹⁸F]Co. No. 1 and [¹⁸F]T807 in a male rhesus monkey.

### DETAILED DESCRIPTION OF THE INVENTION

In an embodiment, the compound of Formula (I), in particular of Formula (I′), is selected from compound 1 (Co. No. 1), compound 2 (Co. No. 2) and compound 3 (Co. No. 3):

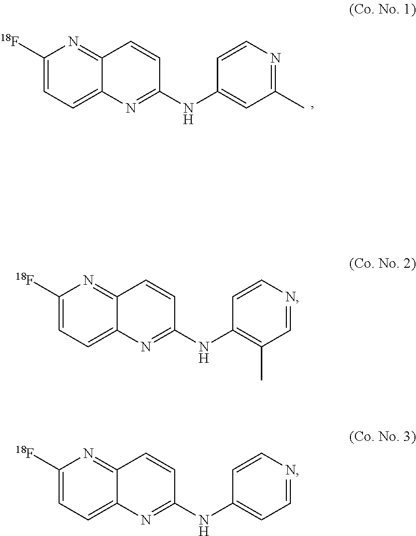 [SMILES: Cc1cc(Nc2ccc3nc([18F])ccc3n2)ccn1 ; Cc1cnccc1Nc1ccc2nc([18F])ccc2n1 ; [18F]c1ccc2nc(Nc3ccncc3)ccc2n1]

or a pharmaceutically acceptable salt or a solvate thereof.

The invention also relates to the reference materials corresponding to the non-radiolabelled compounds 1, 2, and 3, corresponding to the [¹⁹F]-compounds

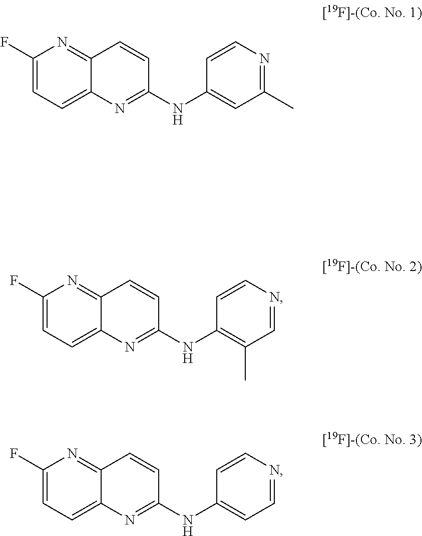 [SMILES: Cc1cc(Nc2ccc3nc(F)ccc3n2)ccn1 ; Cc1cnccc1Nc1ccc2nc(F)ccc2n1 ; Fc1ccc2nc(Nc3ccncc3)ccc2n1]

and the pharmaceutically acceptable salts and the solvates thereof.

[¹⁹F]-Co. No. 1 has shown potent binding (pIC₅₀ 7.7) to extracted human tau in a radiolabel displacement assay using an internally made tritium analogue of compound T-808 (T-808, aka AV-680, CAS [1320211-61-7], 2-[4-(2-fluoroethyl)-1-piperidinyl]-pyrimido[1,2-a]benzimidazole, is developed by Siemens, see for example, J. Alzheimers Dis. 2014, 38, 171-184), and which is referred to herein as [³H]-T808. In addition, Co. No. 1 shows no measurable binding to extracted human amyloid-beta aggregates up to 10 μM in a radiolabel displacement assay using an internally made tritium analogue of Florbetapir (also known as Amyvid® from Eli Lilly and Co., or AV-45, CAS [956103-76-7], (E)-4-(2-(6-(2-(2-(2-fluoroethoxy)ethoxy)ethoxy)pyridin-3-yl)vinyl)-N-methyl benzenamine, see for example, J. Nucl. Med. 2010, 51, 913-920), and which is referred to herein as [³H]-AV-45. A description of the protocols is provided hereinafter.

[¹⁹F]-Co. No. 2 has shown potent binding (pIC₅₀ 7.5) to extracted human tau in a radiolabel displacement assay using [³H]-T808 and no measurable binding to extracted human amyloid-beta aggregates up to 10 μM in a radiolabel displacement assay using [³H]-AV-45.

[¹⁹F]-Co. No. 3 has shown potent binding (pIC₅₀ 7.6) to extracted human tau in a radiolabel displacement assay using [³H]-T808 and no measurable binding to extracted human amyloid-beta aggregates up to 10 μM in a radiolabel displacement assay using [³H]-AV-45.

From the pIC₅₀ values, Kᵢ values can be derived using the Cheng-Prusoff equation (Cheng Y, Prusoff W H (December 1973) Biochem Pharmacol. 22 (23): 3099-108). Summary of binding results for compounds 1 and 2:^{a,b}

| Co. | | Tau Kᵢ | Aβ Kᵢ | Selectivity |
|---|---|---|---|---|
| No. | Structure | (nM) | (nM) | (ratio) |
| 1 | 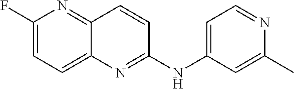 [SMILES: Cc1cc(Nc2ccc3nc(F)ccc3n2)ccn1] | 7.9 ± 2.5 | >4,400 | >540 |
| 2 | 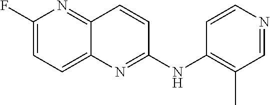 [SMILES: Cc1cnccc1Nc1ccc2nc(F)ccc2n1] | 13.5 ± 2.2 | >4,400 | >325 |
| 3 | 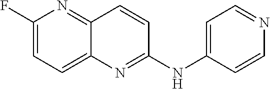 [SMILES: Fc1ccc2nc(Nc3ccncc3)ccc2n1] | 11.3 ± 2.8 | >4,400 | >380 |

^{a} Kᵢ values are a mean of at least two measurements and are given with standard deviation.
^{b}Kᵢ values were calculated from IC₅₀ values using the following equation: Kᵢ = IC₅₀/(1 + (concentration RL/K_{D} RL), with a K_{D} for PHF of 6.275 nM for ³H-AV680, a K_{D} for Aβ of 7.85 nM for ³H-AV45, and 10 nM of RL concentration in both assays.

[³H]-T808 was obtained by subjecting a solution of the bromo precursor (1 eq.) in methanol to catalytic tritiation over palladium on carbon (5%) in the presence of diisopropylethylamine (5 eq.) at room temperature. The bromo precursor was obtained by bromination of T808 with N-bromosuccinimide (1 eq.) in acetonitrile.

[³H]-AV-45 was obtained by Iridium catalyzed (Crabtree's catalyst) tritium exchange of AV-45 dissolved in dichloromethane.

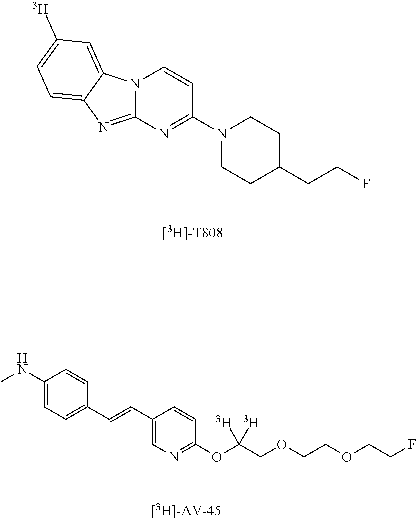 [SMILES: [3H]c1ccc2nc3nc(N4CCC(CCF)CC4)ccn3c2c1 ; [3H]C([3H])(COCCOCCF)Oc1ccc(/C=C/c2ccc(NC)cc2)cn1]

As already mentioned, the compound of Formula (I) and compositions comprising the compound of Formula (I) can be used for imaging a tissue, or a subject, in vitro or in vivo. In particular, the invention relates to a method of imaging or quantifying tau aggregates in a tissue, or a subject in vitro or in vivo.

In particular, the method of imaging tau comprises providing a subject, in particular a patient, with a detectable quantity of a compound of Formula (I).

Further, the invention relates to a method of imaging tau-aggregate deposits comprising the steps of providing a subject with a detectable quantity of a compound of Formula (I), allowing sufficient time for the compound of Formula (I) to be associated with tau aggregate deposits, and detecting the compound associated with tau aggregate deposits.

When the method is performed in vivo, the compound of Formula (I) can be administered intravenously, for example, by injection with a syringe or by means of a peripheral intravenous line, such as a short catheter. The compound of Formula (I) or a sterile solution comprising a compound of Formula (I), may in particular be administered by intravenous administration in the arm, into any identifiable vein, in particular in the back of the hand, or in the median cubital vein at the elbow.

Thus, in a particular embodiment, the invention relates to a method of imaging a subject, comprising the intravenous administration of a compound of Formula (I), as defined herein, or a composition, in particular, a sterile formulation, comprising a compound of Formula (I) to the subject, and imaging the subject with a positron-emission tomography imaging system.

In a further embodiment, the invention relates to a method of quantifying tau aggregation deposits in a subject, comprising the intravenous administration of a compound of Formula (I), or a composition comprising a compound of Formula (I) to the subject, and imaging with a positron-emission tomography imaging system.

The compound is provided to a subject in a detectable quantity and after sufficient time has passed for the compound to become associated with the tau aggregation deposits, the labelled compound is detected noninvasively.

In a further embodiment, the invention relates to a compound (I-6)

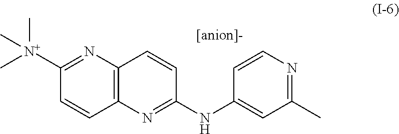

wherein [anion]⁻ is a suitable anionic counterion as defined herein, or a pharmaceutically acceptable salt or a solvate thereof. In a particular embodiment, the anionic counterion is selected from the group consisting of trifluoroacetate (—[OC(O)CF₃]⁻), an organic sulfonate and tartrate, more in particular, trifluoroacetate.

Thus, in a particular embodiment, the invention relates to compound (I-6a)

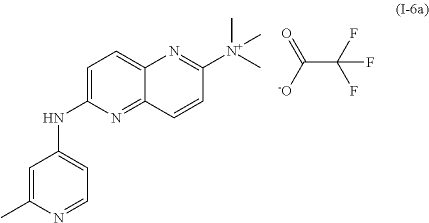

in particular, a trifluoroacetate salt or a solvate thereof, in particular, a hydrate thereof. In an additional embodiment, the invention relates to compound (I-6b)

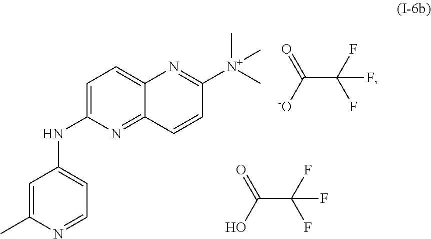

or a solvate thereof, in particular a hydrate thereof.

In an additional embodiment, the invention relates to a compound of Formula (I-6c) or a solvate thereof, in particular, a hydrate thereof

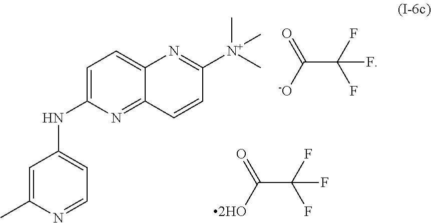

### Definitions

As used herein, the term “composition” is intended to encompass a product comprising the specified ingredients in the specified amounts, as well as any product which results, directly or indirectly, from combinations of the specified ingredients in the specified amounts.

Addition salts of the compounds according to the invention also intended to be encompassed within the scope of this invention.

Acceptable salts of the compounds of the invention are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are included within the ambit of the present invention. The pharmaceutically acceptable salts are defined to comprise the therapeutically active non-toxic acid addition salt forms that the compounds according to the invention are able to form. Said salts can be obtained by treating the base form of the compounds according to the invention with appropriate acids, for example inorganic acids, for example hydrohalic acid, in particular hydrochloric acid, hydrobromic acid, sulphuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzensulfonic acid, p-toluenesulfonic acid, cyclamic acid, salicylic acid, p-aminosalicylic acid and pamoic acid.

Conversely, said salt forms can be converted into the free base form by treatment with an appropriate base.

In addition, some of the compounds of the present invention may form solvates with water (i.e., hydrates) or common organic solvents, and such solvates are also intended to be encompassed within the scope of this invention.

The term “subject” as used herein, refers to a human, who is or has been the object of treatment, observation or experiment. Unless otherwise stated, “subject” includes non-symptomatic humans, presymptomatic humans and human patients.

Preparation

The compounds according to the invention can generally be prepared by a succession of steps, each of which is known to the skilled person. In particular, the compounds can be prepared according to the following synthesis methods.

Compounds of Formula [¹⁹F]—(I) as disclosed herein can be prepared by a reaction of a compound of Formula (I-3) as described herein, with an appropriate 4-amino-pyridine compound of Formula (II) wherein all variables are as described herein for [¹⁹F]—(I)

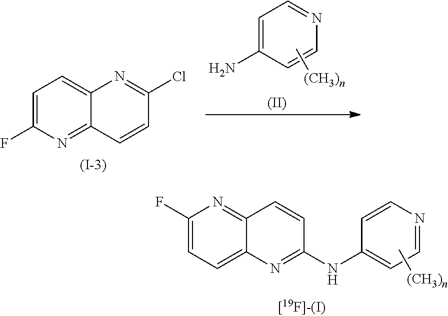

under Buchwald amination conditions.

Compounds of Formula (I′) as disclosed herein can be prepared through the reaction of a compound of Formula (P-1) or (I-A) as defined herein, with a source of fluoride ¹⁸F⁻. Thus, a compound of Formula (I′), or a pharmaceutically acceptable salt or a solvate thereof, as described herein, can be prepared by reaction of a compound of Formula (P-1) or a pharmaceutically acceptable salt or a solvate thereof, as defined herein, wherein in particular, the anionic counterion is trifluoroacetate, with a source of fluoride ¹⁸F⁻ under suitable conditions.

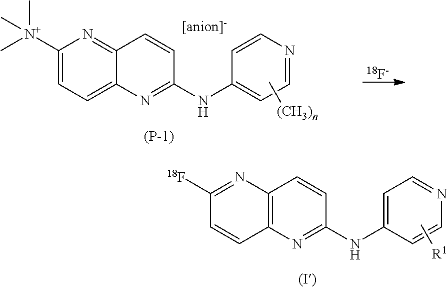

Alternatively, a compound of Formula (I′), or a pharmaceutically acceptable salt or solvate thereof, as described herein, can be prepared by reaction of a compound Formula (I-A) or a pharmaceutically acceptable salt or a solvate thereof, as defined herein, with a source of fluoride ¹⁸F⁻ under suitable conditions.

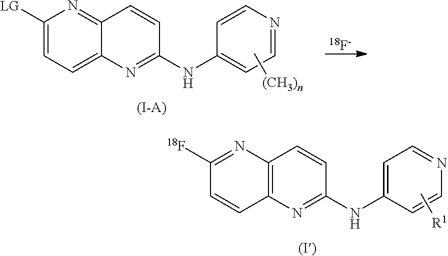

A suitable source of ¹⁸F⁻ is, for example 4,7,13,16,21,24-hexaoxa-1,10-diazabicyclo[8.8.8]hexacosane potassium fluoride-[¹⁸F] (1:1) (also referred to as [¹⁸F]KF.K222). Suitable conditions include, those appropriate for nucleophilic substitution known in the art, for example, using DMF as solvent under conventional heating, for example at about 120° C., for a sufficient period of time to enable the reaction to proceed to completion.

Applications

The compounds according to the present invention find various applications for imaging tissues, or a subject, both in vitro and in vivo. Thus, for instance, they can be used to map the differential distribution of tau aggregate deposits in subjects of different age and sex. Further, they allow one to explore for differential distribution of tau aggregate deposits in subjects afflicted by different diseases or disorders, including Alzheimer's disease, but also other diseases caused by tau aggregate deposits, i.e. other tauopathies.

Thus, excess distribution may be helpful in diagnosis, case finding, stratification of subject populations, and in monitoring disease progression in individual subjects, particularly when anti-tau treatments, e.g. antibodies, become available. Since the radioligand is administered in trace amounts, i.e. in detectable amounts for PET imaging, no therapeutic effect may be attributed to the administration of the radioligands according to the invention.

### EXPERIMENTAL PART

I. Chemistry

As used herein, the term “aq.” means aqueous, “tBuOH” means tert-butanol, “DCM” means dichloromethane, “DIPE” means diisopropyl ether, “DMF” means N,N-dimethylformamide, “Et₂O” means diethyl ether, “EtOAc” means ethyl acetate, “h” means hours, “HPLC” means high-performance liquid chromatography, “LCMS” means liquid chromatography/mass spectrometry, “MeOH” means methanol, “min” means minutes, “m.p.” means melting point, “Pd(OAc)₂” means Palladium(II) acetate, “prep” means preparative, “rm/RM” means reaction mixture, “r.t./RT” means room temperature”, “Rₜ” means retention time (in minutes), “sat.” means saturated, “sol.” means solution, “TBAF” means tetrabutylammonium fluoride, “TEA” means triethylamine, “TFA” means trifluoroacetic acid or trifluoroacetate depending on the context, “THF” means tetrahydrofuran, “XantPhos” means 4,5-bis(diphenyl-phosphino)-9,9-dimethylxanthene.

Thin layer chromatography (TLC) was carried out on silica gel 60 F254 plates (Merck) using reagent grade solvents. Open column chromatography was performed on silica gel, mesh 230-400 particle size and 60 Å pore size (Merck) under standard techniques. Automated flash column chromatography was performed using ready-to-connect disposable cartridges purchased from Grace (GraceResolv™ catridges) or Teledyne ISCO (RediSep catridges), on irregular silica gel, particle size 35-70 μm on an ISCO CombiFlash or Biotage Isolera™ Spektra apparatus.

Nuclear Magnetic Resonance (NMR): For a number of compounds, ¹H NMR spectra were recorded either on a Bruker DPX 360 MHz NMR or Bruker Avance III 400 MHz NMR spectrometer with standard pulse sequences, operating at 360 MHz and 400 MHz, respectively. Samples were dissolved in DMSO-d₆ or CDCl₃ and transferred in 5 mm NMR tubes for the measurement. Chemical shifts (δ) are reported in parts per million (ppm) downfield from tetramethylsilane (TMS), which was used as internal standard.

The values of acid/base stoichiometry and/or water content as provided herein, are those obtained experimentally and may vary when using different analytical methods. For example, the content of trifluoroacetic acid (TFA) reported herein was determined by ¹³C NMR integration, elemental analysis and/or ion chromatography.

Several methods for preparing the compounds of this invention are illustrated in the following examples, which are intended to illustrate but not to limit the scope of the present invention. Unless otherwise noted, all starting materials were obtained from commercial suppliers and used without further purification.

A. Synthesis of Intermediates

Intermediate 1

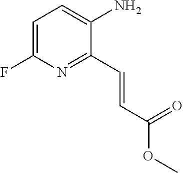

A mixture of 2-bromo-6-fluoro-3-pyridinamine (20 g, 96.33 mmol), methyl acrylate (13.01 mL, 144.50 mmol), Pd(OAc)₂ (2.81 g, 12.52 mmol), PPh₃ (5.81 g, 22.16 mmol) and TEA (30.13 mL, 216.75 mmol) in THF (143 mL) was heated in a sealed tube at 140° C. for 2 h. The reaction was cooled to RT. NaHCO₃ (sat. sol.) and EtOAc were added and the phases were separated. The aqueous phase was extracted twice more with EtOAc. The combined organic layers were dried over MgSO₄, filtered and evaporated. The residue was purified by flash column chromatography (silica; heptane/EtOAc 100/0 to 60/40 (dry loading)). The desired fractions were collected and the solvents were evaporated to yield intermediate 1 (17.6 g, 93%) as an orange solid.

Intermediate 2

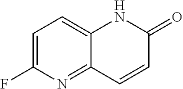 [SMILES: O=c1ccc2nc(F)ccc2[nH]1]

A mixture of intermediate 1 (14.8 g, 75.44 mmol) and tri-n-butylphosphine (18.84 mL, 75.44 mmol) in AcOH (107.87 mL) was heated in a sealed tube at 110° C. for 1 h. The mixture was evaporated and then stirred in DIPE (500 mL). The solids were filtered off and dried under vacuum at 50° C. overnight, yielding intermediate 2 (11.3 g, 91%) as a yellow solid.

Intermediate 3

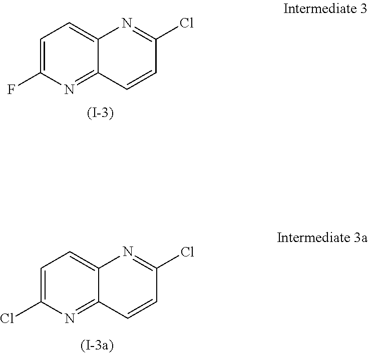

POCl₃ (1.925 mL, 20.71 mmol) was added to a suspension of intermediate 2 (5 g, 20.71 mmol) in 1,4-dioxane (64.32 mL) and the mixture was heated in a pressure tube at 110° C. for 1 h. The RM was evaporated, taken up in a sat. aq. NaHCO₃, extracted with DCM, dried over MgSO₄, and evaporated. The residue was purified by column chromatography (silica gel; heptane/EtOAc 100/0 to 80/20 (dry loading)). The desired fractions were evaporated, yielding intermediate 3 (3.36 g, 89%) as a white solid (the sample was determined to contain 13 mol % intermediate 3a).

¹H NMR (360 MHz, CDCl₃-d) δ ppm 7.35 (dd, J=9.1, 2.9 Hz, 1H) 7.65 (d, J=9.1 Hz, 1H) 8.21 (dd, J=8.8, 0.7 Hz, 1H) 8.36-8.45 (m, 1H).

Intermediate 4

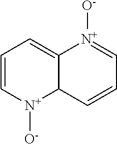 [SMILES: [O-][N+]1=CC=CC2C1=CC=C[NH+]2[O-]]

Procedure a: 3-Chlorobenzoperoxoic acid (70%, 8.33 g, 33.81 mmol) was added to a solution of 1,5-naphthyridine (2 g, 15.37 mmol) in DCM (100 mL). The RM was stirred at RT for 3 h. A precipitate formed, but only still partial conversion was observed. Additional 3-chlorobenzoperoxoic acid (70%, 4.92 g, 19.98 mmol) was added, followed by methanol (50 mL) which dissolved all precipitates, and stirring was continued overnight. Again a solid formed which was filtered. The filtrate was half concentrated and more precipitate formed which was filtered and combined with the precipitate obtained before. This solid was found to contain still 3-chlorobenzoic acid, and was triturated with DCM/MeOH (9:1) for 1 day, filtered and dried in vacuo yielding intermediate 4 (2.18 g, 88%) as a yellow solid.

Procedure b: 3-chlorobenzoperoxoic acid (164.4 g, 666.9 mmol) was added to a solution of 1,5-naphthyridine (28.0 g, 215.1 mmol) in DCM (1.4 L) and MeOH (0.7 L). The reaction mixture was stirred at r.t. for 3 h. More 3-chlorobenzoperoxoic acid (53.0 g, 215.1 mmol) was added, and stirring was continued for 72 h. A solid was formed which was filtered. The filtrate was half concentrated and more precipitate formed which was filtered and combined with the precipitate obtained before. This solid was triturated with DCM/MeOH (9:1), filtered and dried in vacuo yielding intermediate 4 (28.0 g, 172.7 mmol, 80%) as a yellow solid.

Intermediate 5

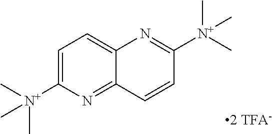

Procedure a: To a stirred solution of intermediate 4 (1 g, 6.17 mmol) and trimethylamine (1 M in THF, 37.02 mL, 37.02 mmol) in DCM (50 mL) was added trifluoroacetic anhydride (2.57 mL, 18.50 mml) at 0° C. The mixture was stirred at RT, and upon completion (about 30 min), Et₂O (50 mL) was added and stirring was continued for 1 h. The white salts were isolated by filtration, washed with Et₂O, and dried in vacuo overnight to yield intermediate 5: trimethylamine.TFA, 40:60 (3.4 g).

Procedure b: To a stirred solution of intermediate 4 (1 g, 6.17 mmol) and trimethylamine (1 M in THF, 30.84 mL, 30.84 mmol) in DCM (59.3 mL) was added trifluoroacetic anhydride (2.57 mL, 18.50 mmol) at 0° C. The mixture was stirred at r.t. for 1 h. Et₂O (50 mL) was added and stirring was continued for 1 h, the white solid was isolated by filtration, washed with Et₂O and dried in vacuo overnight to yield intermediate 5 (1.83 g, 93% pure, 63%) as a white solid.

Procedure c: Trifluoroacetic anhydride (69.5 mL, 499.5 mmol) was added to a stirred solution of intermediate 4 (27.0 g, 166.5 mmol) and trimethylamine (1 M in THF, 0.85 L, 850 mmol) in DCM (1.35 L) at 0° C. The mixture was stirred at r.t. Upon completion (about 30 min), Et₂O (1 L) was added and stirring was continued for 1 h.

The white salts were isolated by filtration, washed with Et₂O, and dried in vacuo overnight to yield intermediate 5 (68 g, 144.0 mmol, 86%).

Intermediate 6a, Intermediate 6B and Intermediate 6C

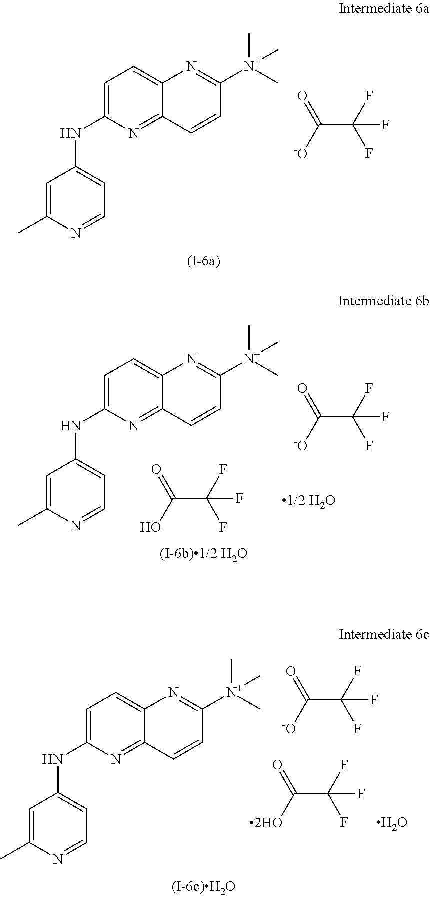

Procedure a: NaH (60% dispersion in mineral oil, 0.614 g, 15.34 mmol) was added to a mixture of intermediate 5 (obtained from procedure a: 40% pure, contains 60% trimethylammonium trifluoroacetate; 1.58 g, 2.56 mmol) and 4-amino-2-methylpyridine (0.277 g, 2.56 mmol) in dry DMF (on molecular sieves, 25.24 mL) at 0° C. The resulting mixture was stirred for 20 min (red color develops) while slowly warming to RT. Silica gel and water (5 mL) were added, the solvent was evaporated to dryness and charged on a flash column (24 g silica), and eluted with 10% MeOH in DCM. The product fractions were evaporated providing 426 mg of a crude, which was again purified by flash column chromatography over 24 g silica, and eluted with 10% MeOH in DCM. Evaporation of the product fractions yielded intermediate 6a (trifluoroacetate salt, 318 mg, 27%, 90% purity; 38 mg, 96% purity) as a yellow sticky solid.

¹H NMR (400 MHz, DMSO-d₆) δ ppm 2.60 (s, 3H) 3.71 (s, 9H) 7.66 (d, J=9.2 Hz, 1H) 8.06 (dd, J=6.2, 1.8 Hz, 1H) 8.19 (s, 1H) 8.37 (d, J=9.2 Hz, 1H) 8.43 (d, J=9.2 Hz, 1H) 8.45 (d, J=6.4 Hz, 1H) 8.71 (d, J=9.2 Hz, 1H) 11.13 (br s, 1H). ¹³C NMR (101 MHz, DMSO-d₆) δ ppm 21.81 (s, 1C), 38.30 (s, 1C), 54.68 (s, 1C), 69.84 (s, 1C), 111.06 (s, 1C), 112.30 (s, 1C), 116.31 (s, 1C), 120.28 (s, 1C), 135.80 (s, 1C), 137.62 (s, 1C), 138.24 (s, 1C), 139.64 (s, 1C), 142.01 (s, 1C), 144.98 (s, 1C), 150.39 (s, 1C), 153.17 (s, 1C), 154.79 (s, 1C), 155.52 (s, 1C), 158.15 (s, 1C), 161.13 (s, 1C). In addition a quartet at δ ppm 157.99 (J=33 Hz) and 117.44 (J=301 Hz) confirmed the presence of CF₃CO₂⁻ as counterion.

Procedure b: This reaction was performed in two separate batches which were combined and worked up together:

Batch 1: NaH (3.81 g, 356 mmol, 60% dispersion in mineral oil) was added to a mixture of intermediate 5 obtained according to procedure c (15.0 g, 31.8 mmol) and 4-amino-2-methylpyridine (3.43 g, 31.8 mmol) in DMF (313 mL, dried on molecular sieves) at 0° C. The resulting mixture was stirred for 20 min (red color develops) while slowly warming to r.t. The solvent was evaporated to dryness and re-dissolved in EtOAc (200 mL) and water (20 mL).

Batch 2: NaH (14.22 g, 356 mmol, 60% dispersion in mineral oil) was added to a mixture of intermediate 5 (56.0 g, 119 mmol) and 4-amino-2-methylpyridine (12.8 g, 119 mmol) in DMF (1170 mL, dried on molecular sieves) at 0° C. The resulting mixture was stirred for 20 min (red color develops) while slowly warming to rt. The solvent was evaporated to dryness and re-dissolved in EtOAc (600 mL) and water (60 mL).

Both solutions were combined, after which the solvent was evaporated again to provide a yellow solid, which was purified by Prep HPLC (Stationary phase: Uptisphere C18 ODB—10 μm, 200 g, 5 cm, mobile phase: 0.1% TFA solution in water+5% MeOH). The RP-HPLC fractions were evaporated to dryness. Ether was added to the resulting oil and slowly evaporated by rotary evaporation (repeated three times), resulting in the formation of yellow crystals. These crystals were triturated with ether overnight until a fine powder was obtained, which was filtered, washed again with ether, and dried overnight in vacuo at 55° C., and then for 24 h in a lyophilizer at r.t. yielding intermediate 6b (double TFA salt and hemihydrate according to elemental analysis, 12.3 g, 23.2 mmol) as a yellow solid.

¹H NMR (600 MHz, DMSO-d₆+C₆D₆, 61° C.) δ ppm 2.65 (s, 3H), 3.71 (s, 9H), 7.82 (d, J=9.1 Hz, 1H), 8.30 (br d, J=5.1 Hz, 1H), 8.36 (d, J=9.1 Hz, 1H), 8.40 (s, 1H), 8.45 (d, J=9.2 Hz, 1H), 8.52 (d, J=6.7 Hz, 1H), 8.69 (d, J=9.2 Hz, 1H), 11.68 (br s, 1H).

Procedure c: NaH (60% dispersion in mineral oil, 4.92 g, 122.98 mmol) was added to a mixture of intermediate 5 (obtained from procedure b: 93% pure, 9.68 g, 20.50 mmol) and 4-amino-2-methylpyridine (2.22 g, 20.50 mmol) in DMF (dry on molecular sieves, 202.3 mL) at 0° C. The resulting mixture was stirred for 20 min (red color developed) while slowly warming to r.t. The solvent was evaporated to dryness, the residue was dissolved in EtOAc then water was added and the mixture was evaporated yielding intermediate 6 as a yellow solid that was purified via Prep HPLC (Stationary phase: Uptisphere C18 ODB—10 μm, 200 g, 5 cm; mobile phase: 0.1% TFA solution in water+5% CH₃CN, MeOH) to provide a solid that was taken up in Et₂O, filtered and then dried in vacuo at 40° C. overnight yielding intermediate 6c (triple TFA salt and monohydrate according to elemental analysis, 2.4 g, 29%) as a yellow solid.

¹H NMR (400 MHz, DMSO-d₆) δ ppm 2.71 (s, 3H) 3.71 (s, 9H) 7.71 (d, J=9.2 Hz, 1H) 8.19 (br d, J=5.1 Hz, 1H) 8.45-8.52 (m, 3H) 8.55 (d, J=7.0 Hz, 1H) 8.81 (dd, J=9.2, 0.7 Hz, 1H) 11.56 (s, 1H).

B. Synthesis of Cold Compounds

Compound [¹⁹F]-1

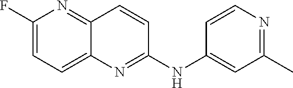 [SMILES: Cc1cc(Nc2ccc3nc(F)ccc3n2)ccn1]

Procedure a: To a solution of 2-methyl-4-pyridinamine (1.66 g, 15.34 mmol) in tBuOH (61.61 mL) were added intermediate 3 (1.66 g, 15.34 mmol), XantPhos (177.47 mg, 0.31 mmol), Pd(OAc)₂ (68.86 mg, 0.31 mmol) and Cs₂CO₃ (13.99 g, 42.94 mmol). Nitrogen was bubbled through the mixture for 5 min, and then the vial was sealed and heated at 140° C. for 18 h. The mixture was then cooled to rt, diluted with water and EtOAc and stirred until most of the solids dissolved. The biphasic mixture was filtered over a glass filter, which was rinsed with EtOAc and water. Next the organic layer was separated and the aqueous layer was extracted (3×) with EtOAc. The combined organic layers were washed with brine, and then dried over MgSO₄, filtered and evaporated. A purification was performed via Prep HPLC (Stationary phase: RP XBridge Prep C18 OBD-10 μm, 50×150 mm; mobile phase: 0.25% NH₄HCO₃ solution in water, MeOH) to yield compound 1 (915 mg, 24%).

Alternatively, the purification was performed by flash column chromatography (silica gel; DCM/7 N NH₃ in MeOH, 100/0 to 90/10). The product fractions were collected and evaporated to dryness, then treated with DIPE and water and the biphasic mixture stirred for 2 h. The resulting crystals were filtered and washed with DIPE and water. After drying in vacuo at 75° C. for 3 h, compound [¹⁹F]-1 was obtained as light yellow crystals.

Procedure b: TBAF (1 M in THF, 0.28 mL, 0.28 mmol) was added to a solution of intermediate 6a (38 mg, 0.093 mmol) in DMF (0.93 mL). The resulting mixture was stirred at 90° C. for 30 min. All volatiles were evaporated. The residue was purified by flash column chromatography over silica gel using a gradient (DCM/7 N NH₃ in MeOH, 1:0 to 0:1). The product fractions were evaporated providing compound [¹⁹F]-1 (15.4 mg, 65%) as yellow crystals.

¹H NMR (400 MHz, DMSO-d₆) δ ppm 2.45 (s, 3H) 7.39 (d, J=9.2 Hz, 1H) 7.48 (dd, J=8.9, 2.8 Hz, 1H) 7.72-7.85 (m, 2H) 8.10 (d, J=9.0 Hz, 1H) 8.28 (d, J=5.7 Hz, 1H) 8.39 (t, J=8.0 Hz, 1H) 9.97 (s, 1H).

Compound [¹⁹F]-2

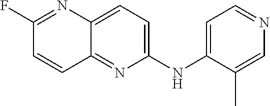 [SMILES: Cc1cnccc1Nc1ccc2nc(F)ccc2n1]

To a solution of 3-methylpyridin-4-amine (118.5 mg, 1.1 mmol) in tBuOH (4.4 mL) were added intermediate 3 (200 mg, 1.1 mmol), XantPhos (12.68 mg, 0.02 mmol), Pd(OAc)₂ (4.9 mg, 0.02 mmol) and potassium phosphate tribasic (651.0 mg, 3.1 mmol). Nitrogen was bubbled through the mixture for 5 min, and then the vial was sealed and heated at 140° C. for 18 h. The mixture was diluted with water and EtOAc and stirred until most of the solids dissolved. The biphasic mixture was filtered over a glass filter, which was rinsed with EtOAc and water. Next the organic layer was separated and the aqueous layer was extracted with EtOAc (3×). The combined organic layers were washed with brine, and then dried (MgSO₄), filtered and evaporated. A purification was performed via Prep HPLC (Stationary phase: RP XBridge Prep C18 OBD-10 μm, 30×150 mm, mobile phase: 0.25% NH₄HCO₃ solution in water, MeOH), the product fractions were evaporated yielding a solid that was re-dissolved in methanol and evaporated again to provide compound [¹⁹F]-2 (37 mg (13%).

¹H NMR (360 MHz, DMSO-d₆) δ ppm 2.32 (s, 3H) 7.46 (dd, J=9.0, 2.7 Hz, 1H) 7.69 (d, J=9.1 Hz, 1H) 8.12 (d, J=9.1 Hz, 1H) 8.27-8.34 (m, 3H) 8.53 (d, J=5.9 Hz, 1H) 8.85 (s, 1H).

Compound [¹⁹F]-3

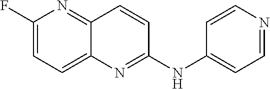 [SMILES: Fc1ccc2nc(Nc3ccncc3)ccc2n1]

To a solution of 4-aminopyridine (114.43 mg, 1.22 mmol) in tBuOH (4.40 mL) were added intermediate 3 (200 mg, 1.10 mmol), XantPhos (12.68 mg, 0.022 mmol), Pd(OAc)₂ (4.92 mg, 0.022 mmol) and Cs₂CO₃ (1 g, 3.07 mmol) and the mixture was heated at 140° C. for 24 h. The mixture was concentrated under reduced pressure, suspended in water, extracted with DCM and EtOAc, dried on MgSO₄, filtered and the solvents evaporated. It was then purified by column chromatography (silica gel, 40 g) using a gradient DCM/NH₃ in MeOH (1:0 to 95:5), providing a yellow solid that was triturated in DIPE and water, filtered and dried in vacuo yielding compound [¹⁹F]-3 (46 mg, 17%).

¹H NMR (360 MHz, DMSO-d₆) δ ppm 7.40 (d, J=8.9 Hz, 1H) 7.49 (dd, J=9.0, 2.7 Hz, 1H) 7.94 (d, =6.2 Hz, 2H) 8.12 (d, J=9.1 Hz, 1H) 8.36-8.45 (m, 3H) 10.10 (s, 1H).

C. Radiosynthesis

Materials and Methods

General

All chemicals and reagents were purchased from commercial sources and used without further purification. [¹⁸F]T807 (aka AV-1451, CAS [1415379-56-4], 7-(6-fluoropyridin-3-yl)-5H-pyrido[4,3-b]indole) was radiolabeled according to a previously reported procedure (Declercq, L. et al. Mol Imaging 2016, 14, 1-15). High-performance liquid chromatography (HPLC) analysis was performed on a LaChrom Elite HPLC system (Hitachi, Darmstadt, Germany) connected to a UV detector set at 254 nm. For analysis of radiolabeled compounds, the HPLC eluate, after passing through the UV-detector, was led over a 3-inch NaI(Tl) scintillation detector connected to a single channel analyzer (GABI box; Raytest, Straubenhardt, Germany). Data were acquired and analyzed using GINA Star (Raytest) data acquisition systems. Quantification of radioactivity in samples of biodistribution (data not shown) and radiometabolite studies was performed using an automated y-counter equipped with a 3-inch NaI(Tl) well crystal coupled to a multichannel analyzer, mounted in a sample changer (Wallac 2480 Wizard 3q, Wallac, Turku, Finland). The values are corrected for background radiation, physical decay and counter dead time. Quantitative data are expressed as mean±standard deviation (SD). Means were compared using an unpaired two-tailed student t-test. Values were considered statistically significant for P≤0.05. Post mortem 10-μm thick human AD brain slices (Braak Stage V-VI) were prepared in house (KU Leuven, Neurology Department, Leuven, Belgium, after approval from the local ethics committee). Animals were housed in individually ventilated cages in a thermo-regulated (˜22° C.), humidity-controlled facility under a 12 h-12 h light-dark cycle, with access to food and water ad libitum. All animal experiments were conducted according to the Belgian code of practice for the care and the use of animals, after approval from the university (KU Leuven) animal ethics committee.

Radiolabeling Co.No. 1 (N=6)

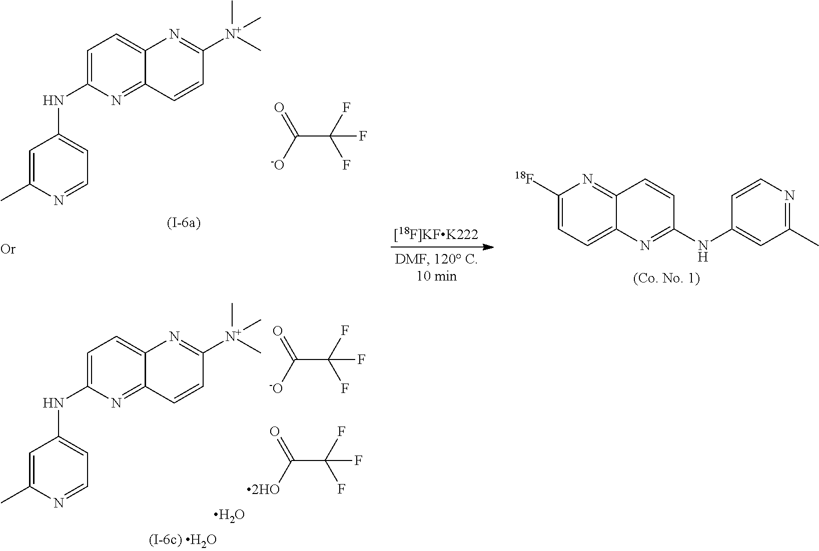

Fluoride-18 ([¹⁸F]F⁻) was produced by an ¹⁸O(p,n)¹⁸F nuclear reaction in a Cyclone 18/9 cyclotron (Ion Beam Applications, Louvain-la-Neuve, Belgium) by irradiation of 2 mL of 97% enriched ¹⁸O—H₂O (Rotem HYOX18, Rotem Industries, Beer Sheva, Israel) with 18-MeV protons. After irradiation, [¹⁸F]F⁻ was trapped on a SepPak Light Accell plus quatemary methyl ammonium (QMA) anion exchange cartridge (CO₃²-form, Waters, Milford, Mass., U.S.A.) and eluted with a mixture of Kryptofix 2.2.2 (K-222, 27.86 mg) and K₂CO₃ (2.5 mg) in CH₃CN/H₂O (0.75 mL; 95:5 v/v). After evaporation of the solvent with a stream of helium at 100° C., anhydrous CH₃CN (1 mL) was added, and [¹⁸F]F⁻ was further dried under the same conditions. A solution of 0.5 mg of the precursor (I-6a or I-6c, respectively, mono- or tris TFA-salts) in 0.3 ml DMF was added to the dried [¹′F]F⁻/K₂CO₃/K-222 residue and the mixture was heated at 120° C. for 10 min (conventional heating). The crude radiolabeling mixture was diluted with 0.6 mL preparative buffer (0.01 M Na₂HPO₄ pH 9.6 and EtOH (65:35 v/v)) and purified using reverse phase HPLC (RP-HPLC) on an XBridge Cis column (5 μm, 4.6 mm×150 mm; Waters, Milford, U.S.A.) eluted with a mixture of 0.01 M Na₂HPO₄ pH 9.6 and EtOH (65:35 v/v) at a flow rate of 0.8 mL/min and with UV detection at 254 nm. The purified radiotracer solution was diluted with saline to obtain an ethanol concentration <10%, suitable for intravenous injection. The solution was subsequently passed through a 0.22-μm filter (Millex-GV, Millipore, Billerica, Mass., U.S.A.) to obtain a sterile product. Quality control was performed using RP-HPLC on an XBridge column (C₁₈, 3.5 μm, 3.0 mm×100 mm; Waters, Milford, U.S.A.) eluted with a mixture of 0.01 M Na₂HPO₄ pH 9.6 and CH₃N (70:30 v/v) at a flow rate of 0.8 mL/min. UV detection was performed at 254 nm.

[¹⁸F]Co. No. 1 was obtained with an average, decay-corrected, radiochemical yield of 46% (relative to radioactivity of [¹⁸F]F⁻ in the preparative chromatogram, n=6). Radiochemical yields were identical for the bis- and tris TFA salt of the precursor of

[¹⁸F]Co. No. 1. The radiochemical purity was examined using HPLC on an analytical C₁₈ column and was more than 98%. [¹⁸F] Co. No. 1 was obtained within a total synthesis time of 60 min, and collected with a specific radioactivity of 65±55 GBq/μmol at the end of synthesis (EOS, n=6).

[¹⁸F]-T807 was collected within a total synthesis time of 60 min with a specific activity of 45±35 GBq/gmol at EOS (n=4) and a radiochemical purity greater than 95%.

II. Analytical Part

LCMS General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector.

TABLE 1
LCMS Method (Flow expressed in mL/min;
column temperature (T) in ° C.; Run time in minutes).

| | | | | Flow | Run |
|---|---|---|---|---|---|
| | | | | — | time |
| Instrument | Column | mobile phase | gradient | Col T | (min) |
| Waters: | Waters: | A: 10 mM | From 100% A to | 0.7 | 3.5 |
| Acquity ® | HSS T3 | CH₃COONH₄ | 5% A in | — | |
| UPLC ®- | (1.8 μm, | in 95% H₂O + | 2.10 min, | 55 | |
| DAD | 2.1*100 | 5% CH₃CN | to 0% A in | | |
| and SQD | mm) | B: CH₃CN | 0.90 min, | | |
| | | | to 5% A in 0.5 | | |
| | | | min | | |

Melting Points

Values are either peak values or melt ranges, and are obtained with experimental uncertainties that are commonly associated with this analytical method.

For a number of compounds, melting points were determined with a DSC823e (Mettler-Toledo). Melting points were measured with a temperature gradient of 10° C./minute. Maximum temperature was 300° C.

TABLE 2
Analytical data

| Co. No. | Rt | MW (theor) | [M + H]⁺ | [M − H]⁻ | MP |
|---|---|---|---|---|---|
| I-6a | 1.00 | 294 | 294 | | |
| I-6b | 1.06 | 294 | 294 | | |
| I-3 | 1.63 | 182 | | | |
| 1 | 1.45 | 254 | 255 | 253 | 246.29 |
| 2 | 1.53 | 254 | 255 | 253 | 210.65 |
| 3 | 1.42 | 240 | 241 | 239 | |

III. Biological Part

Aggregated Tau and Amyloid Plaques Isolation from Human Ad Brain

Enriched aggregated tau fractions were prepared according to a slightly modified version of the protocol described by Greenberg and Davies (Greenberg S G, Davies P. A preparation of Alzheimer paired helical filaments that display distinct tau proteins by polyacrylamide gel electrophoresis. Proc. Natl. Acad. Sci. 1990; 87: 5827-5831) using human AD brain tissue (occipital cortex with high tau fibril load). Briefly, frozen human AD brain samples (˜10 g) were homogenized with 10 vol of cold homogenization buffer (10 mM Tris, 800 mM NaCl, 1 mM EGTA, 10% sucrose, pH 7.4 containing PhosSTOP phosphatase and cOmplete EDTA-free protease inhibitor (Roche, Vilvoorde, Belgium)) on ice. After centrifugation at 27 000×g for 20 min at 4° C. the supernatant was recovered and 1% (w/v) N-lauroylsarcosine and 1% (v/v) 2-mercaptoethanol were added. The N-lauroylsarcosine/2-mercaptoethanol supernatant was incubated for 2 h at 37° C. while shaking on an orbital shaker. Subsequently, ultracentrifugation at 108 000×g for 1.5 h at room temperature enriched aggregated tau in the pellet. Supematant was removed and the pellet was carefully rinsed twice with a small amount of TBS (50 mM Tris, 150 mM NaCl, pH 7.4). Finally, the aggregated tau pellet was recovered in TBS and resuspended to ensure sample homogeneity. Small aliquots were stored at −80° C.

Enriched aggregated β-amyloid preps were prepared from frozen human AD brain samples (10 g—occipital cortex with high amyloid plaques load) that were homogenized with 7-fold vol of cold homogenization buffer (250 mM sucrose, 20 mM Tris base, 1 mM EDTA, 1 mM EGTA and PhosSTOP phosphatase and cOmplete EDTA-free protease inhibitor) on ice. After centrifugation at 27 000×g for 20 min at 4° C. cell debris was removed. Supematant containing amyloid plaques was aliquoted and stored at −80° C.

In Vitro Competitive Radioligand Binding Assays

The competitive radioligand binding assays measure the binding of a radiolabeled reference ligand in the presence of a dose response concentration range of test compounds.

Briefly, aggregated tau preps were diluted to 100 μg protein/ml in PBS buffer with 5% ethanol. In a 96-well format, ³H-T808 (specific activity; 32.97 Ci/mmol) was added at a final concentration of 10 nM to increasing amounts of test compound in the presence of 20 μg protein of aggregated tau prep. Nonspecific binding was defined as the number of counts remaining in the presence of 50 μM Thioflavin T (common beta sheet binder). After 2 h incubation at room temperature, the unbound ligand is removed by filtration of the binding mixtures over GF/B glass filters using a Filtermate 96 harvester instrument (Perkin Elmer, Zaventem, Belgium). The filters were washed three times with PBS buffer containing 20% ethanol. After overnight drying of the filter plate, Microscint O liquid (Perkin Elmer) was added and the amount of radiolabeled ligand bound to the fibrils is measured by liquid scintillation counting in a Topcount instrument (Packard Instrument Company, Connecticut, USA)

Values for half-maximal inhibitory concentration (IC₅₀) were determined from displacement curves of at least two independent experiments using GraphPad Prism software (GraphPad Software, San Diego, Calif.).

To determine compound binding to aggregated β-amyloid a similar assay was put in place but with some minor modifications. Briefly, amyloid preps were diluted to 150 μg protein/ml in 50 mM Tris with 0.1% BSA and 5% ethanol. ³H-AV-45 (florbetapir-specific activity; 45.95 Ci/mmol) was added at a final concentration of 10 nM to increasing amounts of test compound in the presence of 30 μg protein of amyloid plaques prep. Nonspecific binding was determined in the presence of 500 μM Thioflavin T. After 150 min incubation at room temperature, the binding mixtures were filtered over GF/B glass filters. The filters were washed three times with PBS buffer containing 20% ethanol. Subsequent steps were identical to those described for the aggregated tau preps.

[¹⁹F]-Co. No. 2 and [¹⁹F]-Co. No. 3 showed potent binding (pIC₅₀ 7.5 and 7.6, respectively) to extracted human tau using [³H]-T808 and no measurable binding to extracted human amyloid-beta aggregates up to 10 μM using [³H]-AV-45 in this radiolabel displacement assay.

Immunohistochemistry: M&M Human Brain

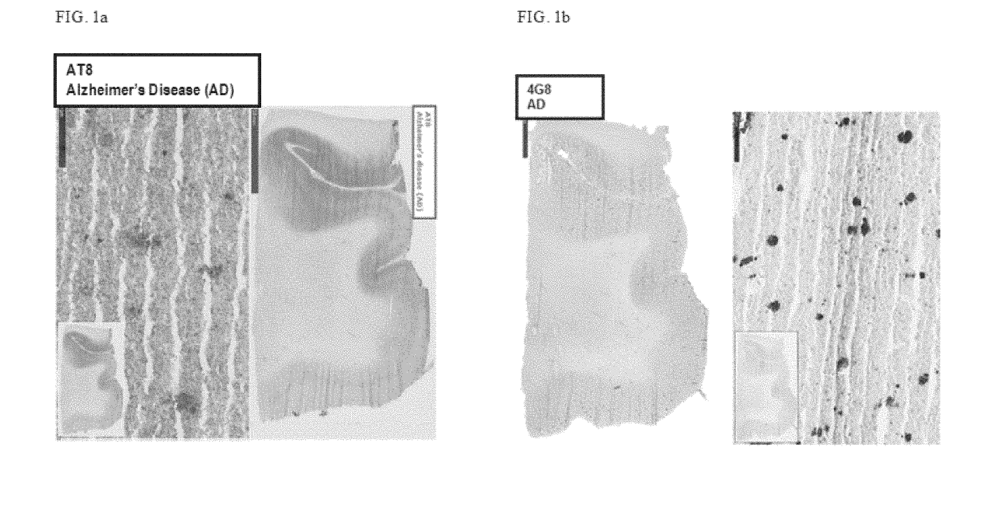

Human AD brain blocks (Braak stage V-VI) were snap-frozen, sliced with a cryostat (20 μm thickness) and stored at −80° C. until used for immunohistochemistry. Sections were dried, fixed in formalin and incubated with hydrogen peroxide (DAKO, S2023) for 5 minutes and blocking reagent (PBS1x+0.05% Triton X-100) during 1 hour. Anti-amyloid or anti-tau antibody [(4G8, Covance, SIG-38220), 1/500 dilution in antibody diluent with background reducing components (DAKO, S3022) or (AT8 (Biema et al., EMBO J. 1992, 11(4): 1593-7), in-house, 1 mg/ml stock concentration), 0.2 μg/mL in antibody diluent with background reducing components (DAKO, S3022)], was applied to the sections for 1 hour. After extensive washing, slides were incubated with HRP-conjugated anti-mouse secondary antibody (Envision, DAKO, K4000), followed by chromogenic DAB labelling (DAKO, K3468). After counterstaining with hematoxylin, sections were dehydrated and mounted with organic mounting medium (Vectamount, Vector labs, H-5000). FIG. 1a shows Tau pathology in AD brain as detected with AT8 IHC and FIG. 1b shows β-amyloid pathology in AD brain as detected with DAKO IHC. The high magnification is taken from the region in the red inset Autoradradiography Studies

Air-dried frozen, 20-μm-thick slices of an AD-patient (Braak stage V-VI) were incubated for 60 min with [¹⁸F]Co. No. 1 (7.4 kBq/500 JpL per section) and subsequently washed with mixtures of phosphate buffered saline (PBS) and ethanol as described elsewhere (Xia C F, Arteaga J, Chen G, et al. [(18)F]T807, a novel tau positron emission tomography imaging agent for Alzheimer's disease. Alzheimers Dement. 2013, 1-11, doi: 10.1016/i.ialz.2012.11.008). To assess specificity of binding, slices were incubated with tracer in the presence of 1 μM of authentic T808 or [¹⁸F]Co.

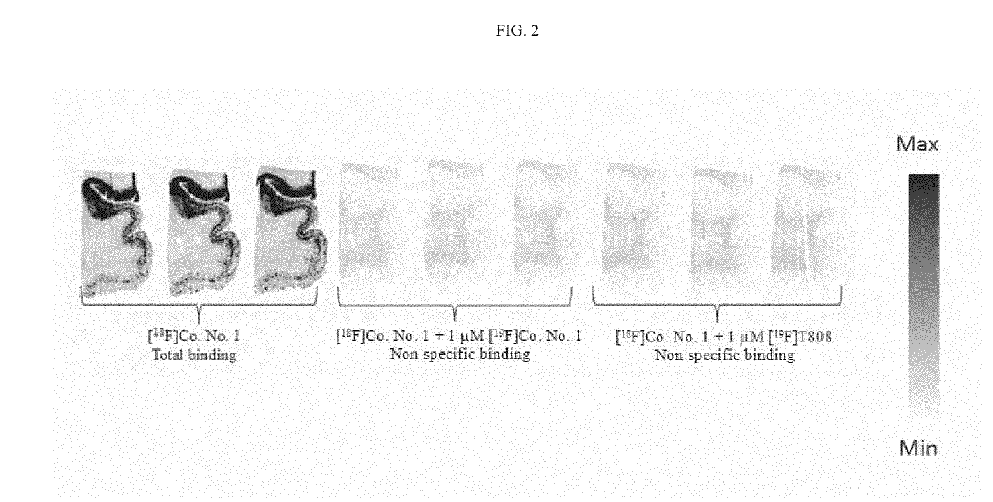

No. 1. After drying, slices were exposed to a phosphor storage screen (super-resolution screen, Perkin Elmer). Screens were read in a Cyclone Plus system (Perkin Elmer, Waltham, Mass., U.S.A.) and analyzed using Optiquant software. Results are expressed as digital light units per square mm (DLU/mm²). Adjacent AD slices were immunostained with anti-tau (AT8) and anti-Aβ antibodies (AG8), to correlate with [¹⁸F]Co. No. 1 binding. FIG. 2 shows binding of [¹⁸F]Co. No. 1 to AD brain section in autoradiography (left). The binding pattern corresponds well with the Tau pathology as detected with AT8 IHC on adjacent slides (see FIG. 1). To assess the specificity of the tracer binding to neurofibrillary tangles (NFTs), blocking studies with authentic Co. No. 1 and T808 were performed. Self-block with 1 j&M of cold Co. No. 1 resulted in 99% inhibition (FIG. 2, centre). Binding of [¹⁸F]Co. No. 1 was reduced with 98% in the presence of 1 lpM T808 (FIG. 2, right). Blocking percentages were calculated as (DLU/mm² in the presence of 1 mol/L blocker)/(DLU/mm² tracer only).

MicroPET Imaging Studies

Wistar Rats

Dynamic 120 min microPET scans were performed on a Focus™ 220 microPET scanner (Concorde Microsystems, Konxville, Tenn., USA) on three female Wistar rats simultaneously, which were kept under gas anesthesia during the whole procedure (2.5% isoflurane in 02 at 1 L/min flow rate). The head of the animals was placed central, in the field of view, of the microPET scanner. Scans were acquired in list mode and acquisition data were Fourier rebinned in 24 time frames (4×15 s, 4×60 s, 5×180 s, 8×300 s, 3×600 s). Data, which were 3D maximum a posteriori (3D-MAP) reconstructed, were manually aligned with a rat brain [¹⁸F]FDG template in Paxinos coordinates using an affine transformation, to allow predefined volumes of interest (VOIs) analysis. Time-activity curves (TACs) of the whole brain were generated using VOIs with PMOD software (v 3.2, PMOD Technologies Ltd., Zdtrich, Switzerland). Radioactivity concentration in the brain was expressed as standardized uptake value (SUV, calculated as (radioactivity in Bq in brain/mL)/(total injected dose (Bq)/body weight in g)) as a function of time after tracer injection. Scans were started immediately after IV injection of 50 MBq [¹⁸F]Co. No. 1 or [¹⁸F]T807 (n=3/tracer). For pre-treatment and displacement studies, cold reference compounds Co. No. 1 or T807 were dissolved in a mixture of 5% DMSO, 5% Tween 80 and 40% (2-hydroxypropyl)-β-cyclodextrin (CD) (prepared as follows: compounds were dissolved in DMSO, then diluted with 40% aqueous CD, followed by addition of 5% aqueous Tween 80 solution (to prevent precipitation), and then further diluted with 40% aqueous CD until a final DMSO concentration of 5%), filtered through a 0.22-μm membrane filter (Millex-GV, Millipore) prior to injection. Pre-treatment (n=1) was done by subcutaneous (SC) injection of 10 mg/kg Co. No. 1 or T807, 60 min prior to radiotracer injection. Displacement (n=1) was performed by IV injection of 1 mg/kg Co. No. 1 or T807, 30 min after radiotracer injection. μPET images were compared to a baseline scan (n=1), acquired in a non-treated rat.

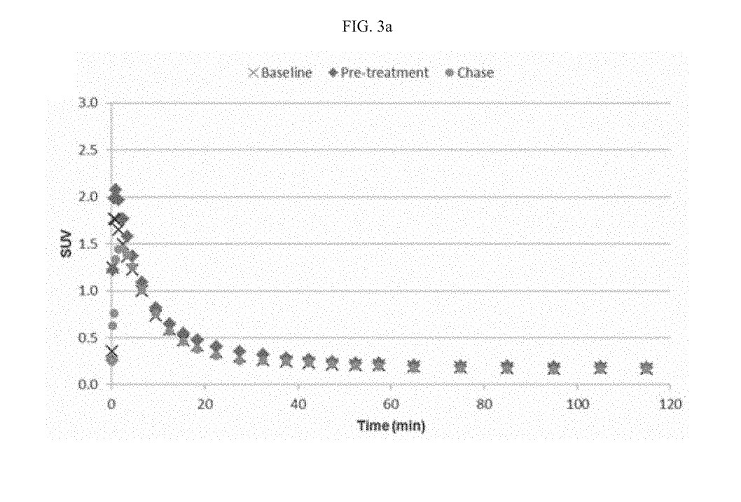

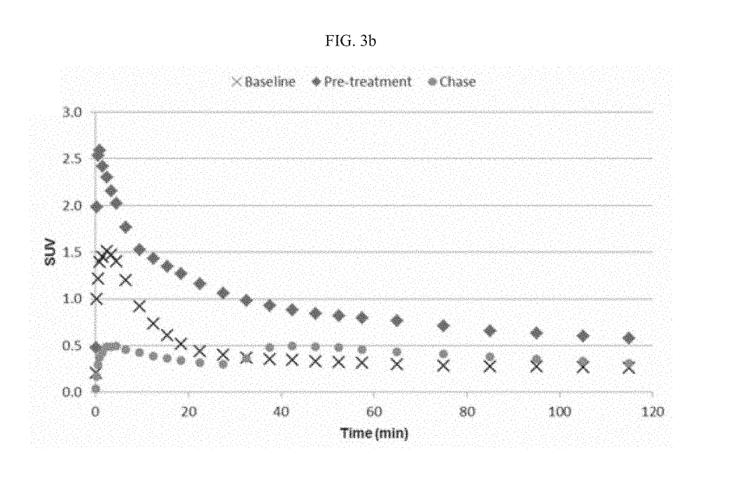

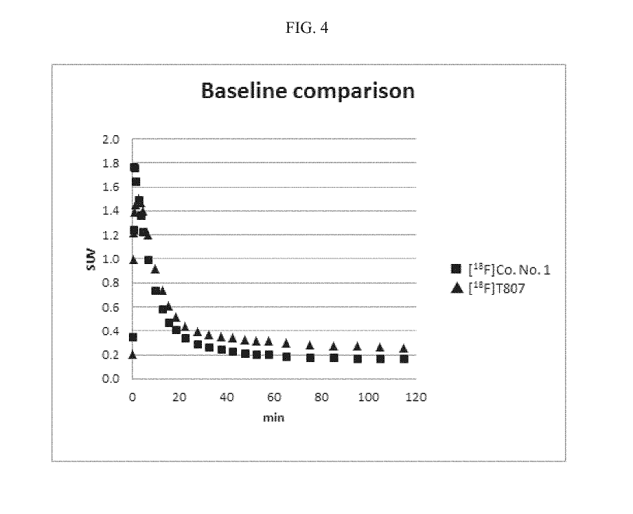

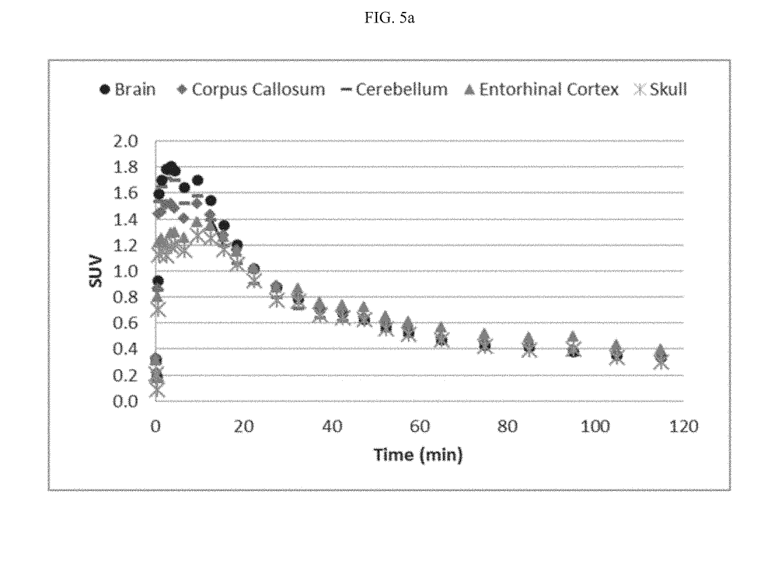

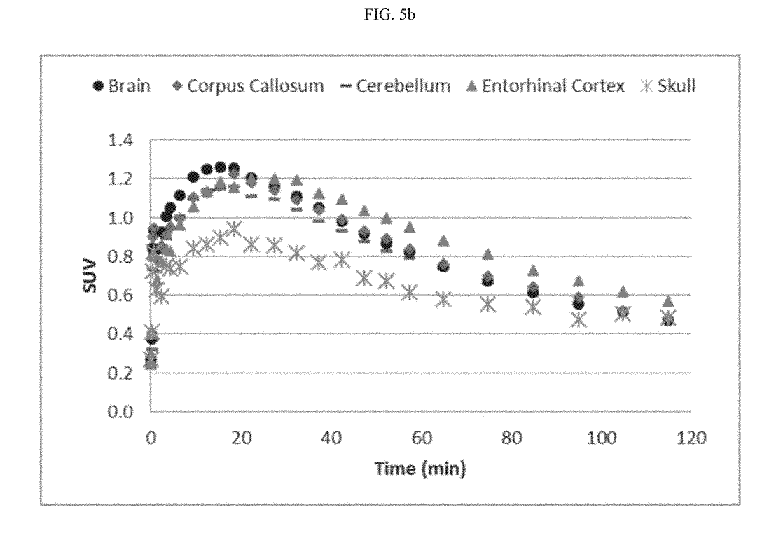

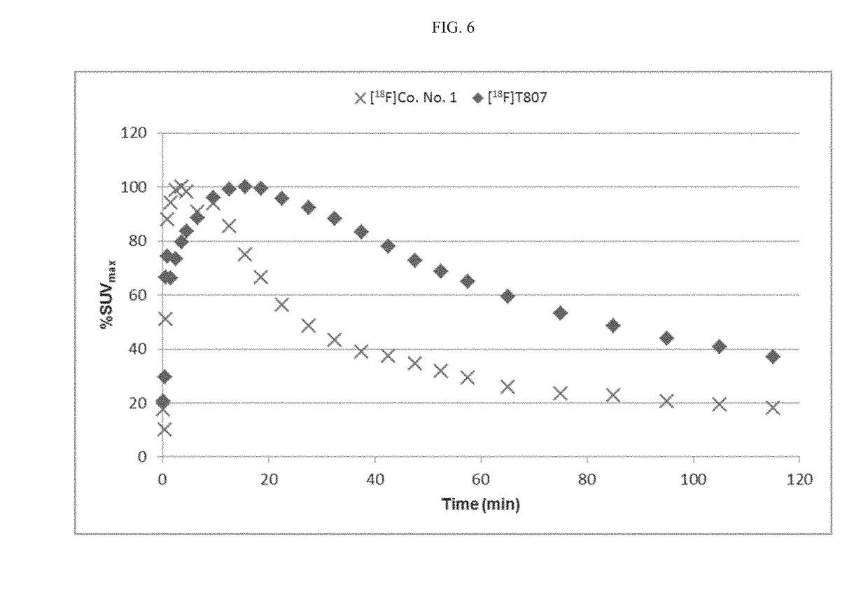

Results of the 120-min baseline, pre-treatment and chase study of [¹⁸F]Co. No. 1 and [¹⁸F]T807 are shown in FIGS. 3a and 3b, respectively (time activity curves, TACs). TACs of the baseline scans of [¹⁸F]Co. No. 1 and [¹⁸F]T807 showed high initial brain uptake, with a comparably high intensity SUV value in the brain of ˜1.6 for both compounds (FIGS. 3 and 4). [¹⁸F]Co. No. 1 had the fastest wash-out rate (SUV value of 0.2 at 60 min p. i.) compared to [¹⁸F]T807 as shown in the SUV curves FIG. 4). No self-blocking or self-chase effect was observed for [¹⁸F]Co. No. 1, indicating absence of specific not tau related binding in brain. Lower brain uptake during the baseline scan of [¹⁸F]T807, compared to the pre-treatment study was recorded. A similar effect was seen 40 min p. i. in the chase study.

MicroPET Imaging Studies

Rhesus Monkey (Experiment 1)

A dynamic 120-min microPET scan with [¹⁸F]Co. No. 1 or [¹⁸F]T807 was performed on a Focus™ 220 microPET scanner (Concorde Microsystems, Knoxville, Tenn., USA) on, respectively, a female rhesus monkey (9 year-old macaca mulatta, 5.3 kg) and a male rhesus monkey (6 year-old macaca mulatta, 7.6 kg), that was sedated with ketamine (Ketalar®) and xylazine (Rompun®) via intramuscular (IM) injection. During scanning the monkey received repeatedly an additional dose of ketamine/xylazine via IV injection. The O₂ saturation in the blood, the breathing frequency and heartrate were monitored during the entire experiment. The head of the animal was placed central, in the field of view, of the microPET scanner. Scans were acquired in list mode and acquisition data were Fourier rebinned in 24 time frames (4×15 s, 4×60 s, 5×180 s, 8×300 s, 3×600 s). Data were reconstructed using a 3D maximum a posteriori (3D-MAP) iterative reconstruction. TACs of the whole brain, corpus callosum, cerebellum and entorhinal cortex were generated using VOIs with PMOD software. Radioactivity concentration in the brain is expressed as SUV as a function of time after tracer injection (FIGS. 5a and 5b). Scans were started immediately after IV injection of 185 MBq of [¹⁸F]Co. No. 1 of [¹⁸F]T807 via the vena saphena of the right leg. Results of the 120-min baseline scan of [¹⁸F]Co. No. 1 and [¹⁸F]T807 are shown in FIGS. 5a and 5b, respectively. Again TACs of the baseline scans of [¹⁸F]Co. No. 1 show high initial brain uptake with a rapid wash-out (SUV value of ˜1.8) and no increased white matter binding was recorded (FIG. 5a). TACs of the baseline scan of [¹⁸F]T807 in the brain show a slower initial brain uptake (SUV value of ˜1.3) and slower wash-out (FIG. 5b). TACs at the side of the skull did not increase as a function of time for both compounds. The foci of high [¹⁸F]Co. No. 1 uptake around the skull are therefore likely due to retention of [¹⁸F]Co. No. 1 to scar or inflammatory tissue resulting from the fixation of an acrylic headpost to the monkey's skull.

Rhesus Monkey (Experiment 2)

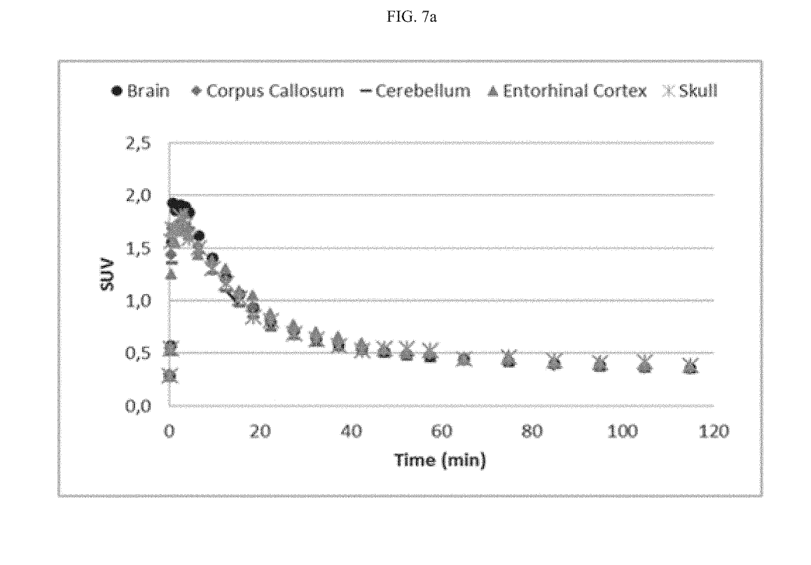

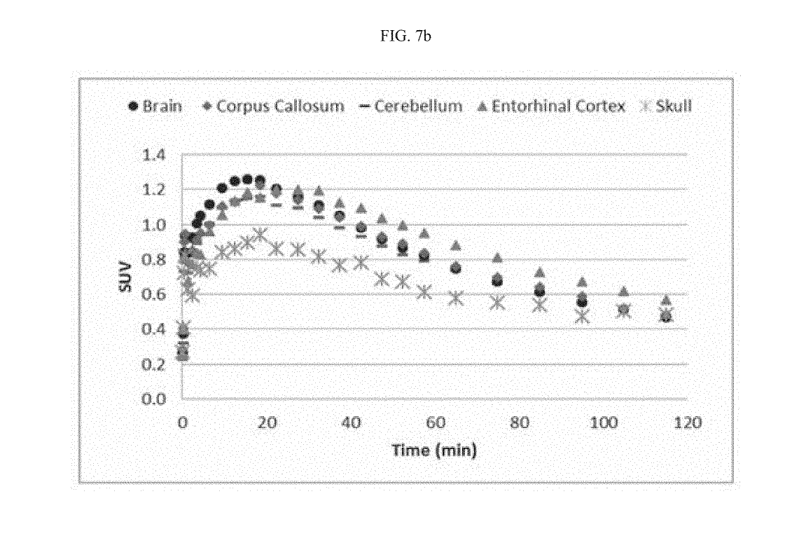

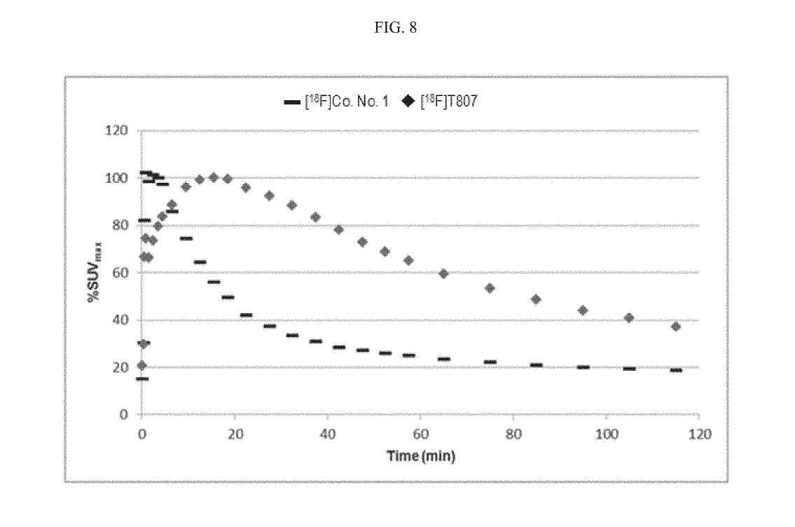

A dynamic 120-min μPET scan with [¹⁸F]Co. No. 1 or [¹⁸F]T807 was performed with a Focus 220 μPET scanner on a male rhesus monkey (6 y-old Macaca mulatta, 7.6 kg), that was sedated with ketamine (Ketalar®) and xylazine (Rompun®) via intramuscular (IM) injection. During scanning the monkey received repeatedly an additional dose of ketamine/xylazine via i.v. injection. O₂ saturation in blood, breathing frequency and heartbeat frequency were monitored during the entire experiment. The head of the animal was placed central in the field of view of the μPET scanner. Scans were acquired in list mode and Fourier rebinned in 24 time frames (4×15 s, 4×60 s, 5×180 s, 8×300 s, 3×600 s). Data were reconstructed using a 3D maximum a posteriori (3D-MAP) iterative reconstruction. TACs of the whole brain were generated using VOIs with PMOD software. Radioactivity concentration in the brain is expressed as SUV as a function of time after tracer injection. Scans were started immediately after i.v. injection of 185 MBq of [¹⁸F]Co. No. 1 or [¹⁸F]T807 via the vena saphena of the right leg. Results of the 120-min baseline scan of [¹⁸F]Co. No. 1 and [¹⁸F]T807 are shown in FIGS. 7a and 7b, respectively. TACs of the baseline scan of [¹⁸F]Co. No. 1 in the brain show a fast high initial brain uptake with a rapid wash-out (SUV value of ˜1.9, time to peak: 1 min) and low white matter binding was recorded. TACs of the baseline scan of [¹⁸F]T807 in the brain show a slower initial brain uptake (SUV value of ˜1.3, time to peak uptake: 15 min) and wash-out (FIG. 8). TACs of the skull show that the SUV signal did not increase as a function of time for both compounds. The foci of high [¹⁸F]Co. No. 1 uptake around the skull observed in experiment 1 were not observed to the same extent in experiment 2. TACs of the skull, of [¹⁸F]Co. No. 1 and [¹⁸F]T807 showed that the focally increased uptake around the skull cannot be attributed to bone uptake, related to [¹⁸F]fluoride, as the signal declines over time.

1. A compound according to Formula (I) 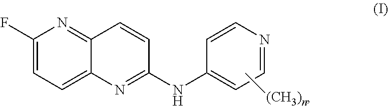
wherein
at least one atom is radioactive, and wherein the methyl substituent when present is bound to any available carbon atom in the pyridyl ring and n is 0 or 1,
or a pharmaceutically acceptable salt or a solvate thereof.

2. The compound according to claim 1, having the Formula (I′) 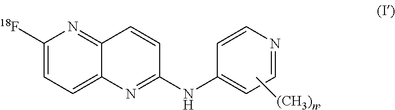
wherein
the methyl substituent when present is bound to any available carbon atom in the pyridyl ring and n is 0 or 1, or a pharmaceutically acceptable salt or a solvate thereof.

3. The compound according to claim 1 or 2, selected from the group consisting of 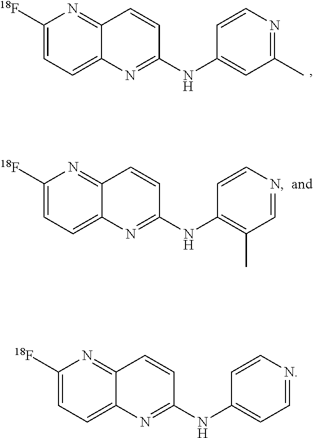
or a pharmaceutically acceptable salt or a solvate thereof.

4. A pharmaceutical composition comprising a compound of claim 1, or a pharmaceutically acceptable salt or a solvate thereof, and a pharmaceutically acceptable carrier or diluent.

5. The pharmaceutical composition according to claim 4, wherein such composition is a sterile solution.

6. A compound of claim 1 for use in binding and imaging tau aggregates.

7. A compound of claim 1, for use in diagnostic imaging of tau aggregates in the brain of a subject.

8. A compound of claim 1, for use in binding and imaging tau aggregates in a patient suffering from, or suspected to be suffering from, a tauopathy.

9. Use of a compound of claim 1, for binding and imaging tau aggregates.

10. A process for the preparation of a compound of claim 1, comprising (a) the step of reacting a compound of Formula (P-1) or a pharmaceutically acceptable salt or a solvate thereof, wherein the methyl substituent when present is bound to any available carbon atom in the pyridyl ring, n is 0 or 1, and [anion]⁻ is a suitable anionic counterion, with a source of fluoride ¹⁸F⁻ under suitable conditions or (b) the step of reacting a compound of Formula (I-A) or a pharmaceutically acceptable salt or a solvate thereof, wherein the methyl substituent when present is bound to any available carbon atom in the pyridyl ring, n is 0 or 1 and LG is a suitable leaving group, with a source of fluoride ¹⁸F⁻ under suitable conditions

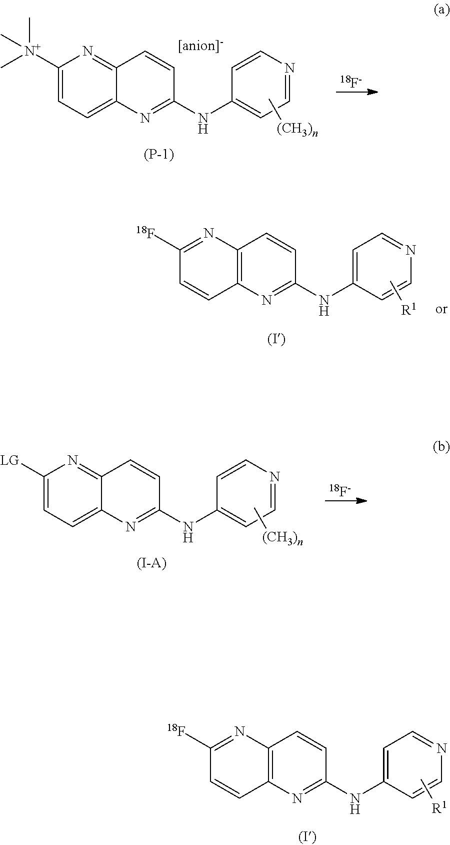

11. A compound selected from Formula (I-A) and (P-1) 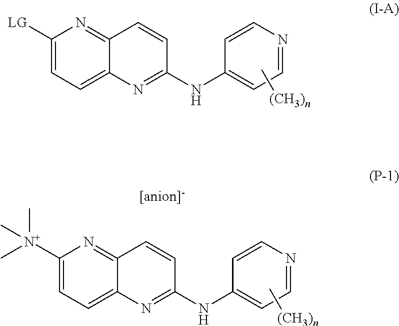
wherein LG is a suitable leaving group, wherein [anion]⁻ is a suitable anionic counterion, and wherein the methyl substituent when present is bound to any available carbon atom in the pyridyl ring, and n is 0 or 1, or a pharmaceutically acceptable salt or a solvate thereof.

12. A compound having the formula [¹⁹F]—(I) 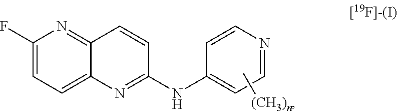
wherein the methyl substituent when present is bound to any available carbon atom in the pyridyl ring and n is 0 or 1, or a pharmaceutically acceptable salt or a solvate thereof.

13. A process for the preparation of the compound defined in claim 12, comprising the step of reacting the compound of Formula (I-3), with an appropriate 4-amino-pyridine compound of Formula (II), wherein the methyl substituent when present is bound to any available carbon atom in the pyridyl ring, and n is 0 or 1 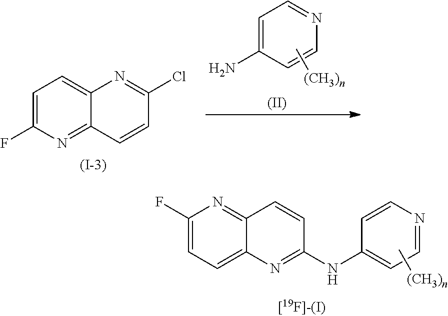
under Buchwald amination conditions.

14. A compound of Formula (I-3) 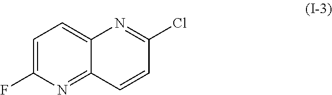 [SMILES: Fc1ccc2nc(Cl)ccc2n1]
or a pharmaceutically acceptable salt or a solvate thereof.
